(19) United States
(12) Patent Application Publication
(10) Pub. No.: US 2009/0254133 A1
(43) Pub. Date: Oct. 8, 2009

(54) Method for treatment of diabetes by electrostimulatioin

(76) Inventor: Keith F. Wendell, North Tamborine (AU)

Correspondence Address: JOHN H. FARO, ESQ., 1581 BRICKELL AVENUE - SUITE 2004, MIAMI, FL, 33129, US

(21) Appl. No.: 12/080,916

(22) Filed: Apr. 7, 2008

(51) Int. Cl. A61N 1/02

(52) U.S. Cl. 607/2

(57) ABSTRACT

A method for treating a patient suffering diabetes or pre-diabetes is disclosed. In brief, this treatment involves delivering a micro-current to the patient, in accordance with a defined therapeutic regime. More specifically, this invention relates to the treatment of diabetes, by means of electrostimulation, so as to correct/abate the biological processes which are believed to be destructive of the cells involved in the production of insulin. In practice a body conformable, electrode wrap is secured to an individual, and an alternating, direct current applied to the electrode wrap, within the range of from 20 milli-amperes to 1 atto-ampere (1×10⁻¹⁸ amps). The micro current is preferably an alternating direct current having a frequency in the range of from 0.00065 Hz to 0.00085 Hz. This therapeutic regime is effective in the stabilization of blood sugar levels in insulin dependent diabetics.

### BACKGROUND OF INVENTION

1. Field Of The Invention

This invention relates to a therapeutic method. More specifically, this invention relates to the treatment of diabetes by means of electrostimulation, so as to correct/abate the biological processes which are believed to be destructive of the cells involved in the production of insulin.

2. Description Of The Prior Art

There are an estimated 18.2 million people in the United States, or 6.3% of the population, who have diabetes. While an estimated 13 million of these people have been diagnosed with diabetes, it is believed that about 5.2 million people are unaware that they have the disease. Diabetes is the fourth most common reason for patient contact with the physician, accounting for approximately 12% of US healthcare dollars, and a total annual cost exceeding $100 billion. Worldwide, diabetes affects more than 135 million people.

There are a number of recognized forms of diabetes, including Type 1 diabetes mellitus, which is characterized by loss of the insulin-producing beta cells of the islets of Langerhans in the pancreas, leading to a deficiency of insulin. The main cause of this beta cell loss is a T-cell mediated autoimmune attack. There is no known preventative measure that can be taken against Type 1 diabetes, which comprises up to 10% of diabetes mellitus cases in North America and Europe (though this varies by geographical location). Most affected people are otherwise healthy and of a healthy weight when onset occurs. Sensitivity and responsiveness to insulin are usually normal, especially in the early stages. Type 1 diabetes can affect children or adults but was traditionally termed “juvenile diabetes” because it represents a majority of cases of diabetes affecting children.

The principal treatment of Type 1 diabetes, even from the earliest stages, is replacement of insulin, combined with careful monitoring of blood glucose levels using blood testing monitors. Without insulin, diabetic ketoacidosis can develop, and may result in coma or death. Emphasis is also placed on lifestyle adjustments (diet and exetcise) though these cannot reverse the loss. Apart from the common subcutaneous injections, it is also possible to deliver insulin by a pump, which allows continuous infusion of insulin 24 hours a day at pre-beta cell levels, and the ability to program doses of insulin as needed at meal times. An inhaled form of insulin, Exubera, was approved by the FDA in January 2006, although Pfizer discontinued Exubera in October 2007.

Type 1 treatment must be continued indefinitely. Treatment does not impair normal activities, if sufficient awareness, appropriate care and discipline in testing and, medication is taken. The average glucose level for the Type 1 patient should be as close to normal, (80-120 mg/dl, 4-6 mmol/l), as possible. Some physicians suggest up to 140-150 mg/dl (7-7.5 mmol/l) for those having trouble with lower values, such as frequent hypoglycemic events. Values above 200 mg/dl (10 mmol/l) are often accompanied by discomfort and frequent urination leading to dehydration. Values above 300 mg/dl (15 mmol/l) usually require immediate treatment and may lead to ketoacidosis. Low levels of blood glucose, called hypoglycemia, may lead to seizures or episodes of unconsciousness. It is well established that most life threatening consequences of diabetes are heart disease and strokes, as well as chronic rendal failure. The American Diabetic Association states that people in the pre-diabetes stage can prevent the development of Type 2 diabetes by making changes in their diet and by increasing their level of physical activity.

Hyperglycemia causes many of the pathological consequences of both Type 1 and Type 2 diabetes. The association between hyperglycemia and oxidative damage to the insulin producing cells in the pancreas has been noted for sometime. It has been suggested that increasing mitochondrial reactive oxygen species production during hyperglycemia may be a predisposing factor of diabetes. Mitochondrial function is particularly susceptible to oxidative damage, leading to decreased mitochondrial ATP synthesis, cellular calcium, dyshomeostasis, and induction of the mitochondrial permeability transition, all of which dispose cells to necrosis or apoptosis.

Mitochondrial reactive oxygen species production, associated with hyperglycemia, also disrupts glucose-stimulated insulin secretion by β-cells, because pancreatic β-cells are particularly susceptible to oxidative damage. Therefore, it is believed that mitochondrial reactive oxygen species production, and oxidative damage, may contribute to the onset, progression and pathological consequences of both Type 1 and Type 2 diabetes.

The mitochondrial respiratory chain is the major site of reactive oxygen species production within the cell. Superoxide is thought to be produced continually as a by-product of normal respiration through the one-electron reduction of molecular oxygen. Consequently, mitochondrial superoxide production initiates a range of damaging reactants through the production of superoxide, hydrogen peroxide, ferrous iron, hydroxyl radical, and peroxynitrite which can damage lipids, proteins and nucleic acids.

There is presently no effective way to abate/modulate the mitochondrial chain to prevent the production of reactive oxygen species, which can impair insulin production and cause irremediable damage to the pancreas. Accordingly, individuals with Type 1 diabetes face a life-long dependence on daily testing, dietary restrictions and dependence upon a daily supplementation of insulin.

### OBJECTS OF THE INVENTION

It is the object of this invention to remedy the above, as well related deficiencies in the prior art.

More specifically, it is the principle object of this invention to provide a method for the treatment of diabetes, specifically, Type 1 & Type 2 diabetes, by means of electrostimulation therapy.

It is another object of this invention to provide a method for the treatment of Type 1 & Type 2 diabetes in individuals, by delivering a mirco current to such individual in accordance with a defined treatment regime.

It is yet another object of this invention to provide a method for the treatment of Type 1 & Type 2 diabetes in individuals, by delivering a mirco current to such individual in accordance with a defined treatment regime, so as to repair damage to insulin producing cells caused by reactive oxidative species, e. g. free radicals, superoxides, etc.

It is still yet another object of this invention to provide a method for the treatment of Type 1 & Type 2 diabetes in individuals, by delivering a mirco current to such individual in accordance with a defined treatment regime, so as to abate damage to insulin producing cells caused by reactive oxidative species, e. g. free radicals, superoxides, etc.

Still yet additional objects of this invention include the provision of a method for the treatment of Type 1 & Type 2 diabetes in individuals, by disruption in the auto-immune cascade, which causes damage to insulin producing cells, by delivering a mirco current to such affected individuals, in accordance with a defined treatment regime,

### SUMMARY OF THE INVENTION

It is the object of this invention to remedy the above as well as related deficiencies in the prior art. More specifically, it is the principle object of this invention to treat an individual afflicted with diabetes, including specifically, Type 1 & Type 2 diabetes, by abatement in the biologically destructive processes, which causes damage to insulin producing cells. The treatment regime of this invention generally includes initially determining the level of glycolated hemoglobin (HBA1c) in the individual's blood, and thereafter subjecting the individual to a micro-current therapy, in accordance with a defined regime. The treatment regime can, and preferably includes, a daily micro-current treatment, which comprises a progressive increase in the current, applied to the individual over the duration of a given treatment cycle.

Once the initial basal level of the foregoing substances is determined, a body conformable electrode wrap is applied to the patient, and a therapeutic effective amount of alternating direct current applied to such electrode over a specified period of time. In the preferred embodiments of this invention, the individual it treated with a micro-current within the range of from 20 milli-amperes to about 1 atto-ampere (1×10⁻¹⁸ amps). In the most preferred embodiments of this invention, the micro current is in the range from about 3 milli-amperes to about 100 atto-amperes. The micro current is preferably an alternating, direct current having a frequency of 0.00065 Hz to 0.00085 Hz.

The preferred delivery system for treatment of an individual afflicted with diabetes, in accordance with the regime of this invention, comprises an electrostimulation system of the type described in co-pending, commonly assigned, U.S. patent application Ser. No. 09/656519 filed Sep. 7, 2000, now U.S. patent Ser. No. ______; and, International Patent Application No. PCT/US2001/042099, EP Reference No. 2001975744, Granted Nov. 28, 2007, which are herein incorporated by reference in its entirety.

In accordance with this system a therapeutic effective amount of alternating direct current delivered to the patient, over a specified period of time. In the preferred embodiments of this invention, the therapeutic treatment is delivered with a BodiHealth TENS System, (commercially available from EPRT Technologies, Simi Valley, Calif., USA). This system utilizes a body conformable electrode wrap for delivery of the micro current to the patient. The body conformable electrode wrap can be applied to the patient's torso, his neck or to his head. The alternating direct current is supplied to the electrode wrap by a current output generator (COG). The alternating direct current supplied to this electrode wrap is preferably in the range of from 20 milli-amperes to 1 atto-ampere (1×10⁻¹⁸ amps), and most preferably in the range of from 3 milli-amperes to 100 atto-amperes; and at a frequency in the range of from 0.00065 Hz to 0.00085 Hz. A duty cycle of 0.000732 Hz, or one cycle about every 25 minutes, has been shown to be found to be efficacious in the treatment regimes of this invention.

### BRIEF DESCRIPTION OF THE INVENTION INCLUDING PREFERRED EMBODIMENTS

The mechanism of insulin release in normal pancreatic beta cells is well-known. Insulin production is more or less constant within the beta cells, irrespective of blood glucose levels. It is stored within vacuoles pending release, via exocytosis, which is triggered by increased blood glucose levels. Insulin is the principal hormone that regulates uptake of glucose from the blood into most cells (primarily muscle and fat cells, but not central nervous system cells). Therefore, a deficiency of insulin, or the insensitivity of its receptors, plays a central role in all forms of diabetes mellitus.

Insulin is released into the blood by beta cells (0-cells), found in the Islets of Langerhans in the pancreas, in response to rising levels of blood glucose after eating. Insulin is used by about two-thirds of the body's cells to absorb glucose from the blood for use as fuel, for conversion to other needed molecules, or for storage. Insulin is also the principal control signal for conversion of glucose to glycogen for internal storage in liver and muscle cells. Lowered glucose levels result both in the reduced release of insulin from the beta cells, and in the reverse conversion of glycogen to glucose when glucose levels fall.

Higher insulin levels increase many anabolic (“building up”) processes such as cell growth and duplication, protein synthesis, and fat storage. Insulin (or its lack) is the principal signal in converting many of the bidirectional processes of metabolism from a catabolic to an anabolic direction, and vice versa. In particular, a low insulin level is the trigger for entering or leaving ketosis (the fat burning metabolic phase).

If the amount of insulin available is insufficient, if cells respond poorly to the effects of insulin (insulin insensitivity or resistance), or if the insulin itself is defective, then glucose will not be absorbed properly by those body cells that require, nor will it be stored appropriately in the liver and muscles. The net effect is persistent high levels of blood glucose, poor protein synthesis, and other metabolic derangements, such as acidosis.

The therapeutic method of this invention provides a non-invasive, treatment regime for control/abatement in the biologically destructive processes, which causes damage to insulin producing cells in individuals suffering from Type 1 and Type 2 diabetes. The therapeutic method contemplates the utilization of a system for delivery of a micro-current to an individual afflicted with diabetes, by means of body conformable electrode wrap, which is electrically coupled to a current output generator.

The body conformable electrode wrap can be placed on the individual's torso, and a micro current delivered to the individual in accordance with a defined treatment regime. In the preferred embodiments of this invention, the wrap is comprised of 3 layers; an open cell foam layer (medi-sponge 100 manufactured by Lindell Manufacturing), which is moisture absorbing, an electrically conductive layer composed of a rip stop nylon silver plated fabric produced by Argentum Medical, and an outer layer comprised of a polyurethane clear plastic layer. The foam or moisture absorbing layer is laminated to conductive layer, such that the foam does not seal solidly against the fabric, but leaves conductive paths for water through the open cells to foam an electrical path from the fabric through the water in the foam. The polyurethane is laminated to the free surface/opposite side of the silver fabric to prevent wicking of moisture from the foam by clothing or linen and also to minimize evaporation.

In use, the foam layer of the wrap is lightly saturated with tap water and wrap is placed around the appropriate body part. An electrically conductive hook and loop fastener is utilized for mechanically fastening the wrap around and to itself, and also completes a circular electrical path around the body parts. Non-electrically conductive hook and loop may be used for fastening the wrap; however, it will reduce the effectiveness. Alternatively, a small strip of conductive hook and loop Velcro may be used with nonconductive hook and loop Velcro, particularly on wider wraps. Metal snaps are then coupled to conductive elements of the wrap for making a connection to the instrument output leads (current generator). It is believed that the effectiveness of the wrap is due, in part, to its height above the skin, the typical total thickness of a wrap is ˜0.25 inch. This causes the electrical flow to go through the energy field just above the skin, as well as through the conductive body meridians.

In order to confirm the efficacy of the treatment and/or to adjust the treatment regime, blood samples are periodically taken from the individual and analyzed—the analytes on interest being indicative of blood sugar level (BSL), and glycated hemoglobin (HbA1c). RbA1c is a measure of the mean blood glucose level over the previous 2-3 months and particularly the previous 4 weeks. HbA1c provides the essential baseline measure of glycemic control in a diabetic, and should be measured at least annually in all diabetics and more often (e.g. bi-monthly), when assessing the affect of changes in therapy or compliance.

The therapeutic regime of this invention is intended to address the problems associated with deficient insulin concentration in the blood. More specifically, the micro current that is delivered to the individual, is directed to the biological processes that contribute to such deficiency. This micro-current treatment is delivered to the individual through an electrode wrap. The micro-current is preferably within the range of from about 20 milli-amperes to about 1 atto-ampere (1×10⁻¹⁸ amps); and, most within the range from about 3 milli-amperes to about 100 atto-amperes. The micro current may be an alternating current of very low frequency. For example, the micro current may have a frequency of 0.00065 Hz to 0.00085 Hz. The micro current may suitably have a duty cycle of 0.000732 Hz, or one cycle every 22.77 minutes.

Commercially available medical devices suitable for delivery of a therapeutic effective amount of micro-current to a patient afflicted with diabetes are available from EPRT Technologies, Inc., Simi Valley, Calif. The BodiHealth TENS System available from EPRT Technologies, is a non invasive, therapeutic device that delivers ultra-low energy compatible with the natural electric currents of the human body, correcting any imbalance that is present. BodiHealth TENS System is capable of delivery of an alternating current ranging from 100 nanoamperes (10⁻⁹) to 3 milliamperes (10⁻³). The BodiHealth TENS System has U.S. FDA 510(k) clearance to market in the U.S., and is approved for temporary symptomatic relief and management of chronic intractable pain, and as an adjunctive treatment in the management of post-surgical and post-traumatic pain. The BodiHealth TENS System utilizes a body conformable electrode wrap to deliver the current to the patient, and tap water in the pad to channel current from the electrode wrap to the patient.

Without wishing to be bound by theory, it has hypothesized that treating a patient with a micro current, in accordance with the defined regime of this invention, creates a powerful antioxidant affect within tissues and cells, thereby restoring electrochemical balance, in for example, the pancreas, which in turn results in the mitochondria producing a full complement of ATP. As is appreciated the ATP energizes these pancreatic cells, thus, allowing cells within the body to work to their maximum efficiency, promoting circulation and dramatically increasing the healing response within the body. It has been surprisingly discovered that a low concentration steady stream electron flow, produced in the foregoing manner, acts as a highly potent antioxidant that can be focused on the pancreas to thereby treat or ameliorate diabetes.

In some embodiments of the present invention, treatment of a patient may comprise applying one or more cycles of a current of 2 milliamps, with each cycle lasting for approximately 25 minutes. Further cycles may be conducted at 300 nanoamps. The electric current may be applied to any part of the body while still obtaining the beneficial outcomes of the present invention.

### EXAMPLES

The Examples which follow further define, describe and illustrated the preferred methods of this invention. Parts and percentages appearing in such Examples are by weight unless otherwise indicated. Apparatus used in the performance of the preferred methods, and in the analysis for the blood constituents of biological fluid, are also standard, unless otherwise specified. The electrostimulation equipment used in this treatment is specified in these Examples.

### Example 1

Initial Patient Status—A 74 year old woman diagnosed with Hypertension, Type 2 Diabetes, Peripheral Vascular Disease, Hemi forefoot Amputation. She started her treatment on the BodiHealth TENS System, (commercially available from EPRT Technologies, Simi Valley, Calif., USA), on the 4^{th} of Feb. 2005.

Ever since then she has received daily treatments on the BodiHealth TENS System. As a result of such treatment with the BodiHealth TENS System, her blood sugar level (BSL) control improved. She has since reduced her medication and eventually ceased taking any medication for Diabetes on the 7^{th} of Oct. 2005, without any recurrent symptoms.

In this example, progress was measured both in stabilization of the patient's blood sugar level, and also as a function of a reduction in glycated hemoglobin HbA1c. HbA1c is a measure of the mean blood glucose level over the previous 2-3 months, and particularly the previous 4 weeks. It provides the essential baseline measure of glycemic control in a diabetic, and should be measured at least annually in all diabetics, and more often (tri-monthly) when assessing the effect of changes in therapy or compliance. The following scale of values indicates the level (%) of HbA1c for healthy and for diabetic individuals

| Scale for HbA1c. | HbA1c ref. ranges: % |
|---|---|
| Non-diabetic range: | 4-6 |
| Diabetic range: | |
| excellent control | 6-7 |
| good control | 7-8 |
| indifferent control | 8-9 |
| poor control | 9-10 |
| exceptionally poor control | >10 |

The following table depicts the patient's progress as a measure/percentage of HbA1c.

| Date | HbA1c | Currents used and Frequency of treatment |
|---|---|---|
| 15^{th} January 2005 | 9.8 | No Treatment. |
| 15^{th} February 2005 | 7.6 | Started treatment 4^{th} February 2005. |
| | | Currents received; 1 milliampere 23 |
| | | minutes, 400 nanoamperes 138 minutes. |
| | | She received daily treatments. |
| 4^{th} April 2005 | 6.5 | Treatment protocol as above. |
| 30^{th} September 2005 | 5.9 | Treatment protocol as above. |
| August 2006 | 5.8 | Treatment protocol as above. |
| | | No diabetic medication since 7^{th} October |
| | | 2005 |
| February 2007 | 6.1 | Treatment protocol as above. |
| June 2007 | 5.5 | Treatment protocol as above. |

### EXAMPLE 2

Initial Patient Status—A 53 year old woman, who was a Type 1, or insulin dependant diabetic (IDDM). She suffered renal failure, as a result of her diabetes and underwent a kidney and pancreatic transplant in 1994. She has also had a coronary artery bypass graft, and suffers with hypercholesterolemia, left ventricular failure, and renal failure.

She started receiving treatment on the BodiHealth TENS System in February 2006, and has continued mostly uninterrupted with daily treatments. While she is not considered to currently have diabetes, her HbA1c dropped over the time period she was receiving treatments. This was matched by her BSL which also stabilized while was receiving treatment over this period of time.

| Date | HbA1c | Currents used and Frequency of treatment |
|---|---|---|
| 8^{th} | 5.4 | Started treatment 7^{th} February. |
| February | | Currents received; 1 milliampere 23 minutes, 2 |
| 2006 | | milliamperes 23 minutes, 3 milliamperes 23 |
| | | minutes, 80 microamperes 69 minutes, 400 |
| | | nanoamperes 69 minutes. |
| | | Treatments 5 days per week. |
| 27^{th} | 5.3 | Treatment regime as above. |
| February | | |
| 2006 | | |
| 6^{th} March | 5.5 | Treatment regime as above. |
| 2006 | | |
| 27^{th} March | 5.3 | Currents received; 1 milliampere 23 minutes, 2 |
| 2006 | | milliamperes 23 minutes, 80 microamperes 46 |
| | | minutes, 600 nanoamperes 69 minutes. |
| | | Treatments 5 days per week. |
| 3^{rd} May | 5.2 | Currents received; 1 milliampere 23 minutes, |
| 2006 | | 80 microamperes 69 minutes, 600 |
| | | nanoamperes 115 minutes. |
| | | Treatments 5 days per week. |
| 15^{th} May | 5.1 | Treatment regime as above. |
| 2006 | | |

| Time Period | average BSL | n |
|---|---|---|
| Feb-Mar 2006 | 6.6 | 20 |
| Mar-May 2006 | 5.8 | 36 |
| May-Jun 2006 | 5.4 | 15 |

### EXAMPLE 3

Initial Patient Status—A 32 year old Type 1 or insulin dependant diabetic (IDDM). She did not have any other concurrent health problems.

She received 8 treatments over a two week period on the BodiHealth TENS System. Over this time period her actrapid (Insulin) requirement dropped dramatically, and her HbA1c level also came down.

| Date | HbA1c | Currents used and Frequency of treatment |
|---|---|---|
| May 2005 | 8.1 | No Treatment. |
| October | 7.1 | Treatments started in end of September. |
| 2005 | | Currents received; 1 milliampere for 23 minutes, |
| | | 60 microamperes for 23 minutes, 400 |
| | | nanoamperes 138 minutes. |
| | | She had 8 treatments over a 2 week period |

### EXAMPLE 4

Initial Patient Status—A 59 year old woman who had Type 2 diabetes, hypertension, fibromyalgia, chronic active hepatitis, and Bowens disease.

She initiated her treatments in October 2006 and had daily treatments through December 2006, on the BodiHealth TENS System. She then had 3 treatments per month through until March 2007, and no treatments though until June 2007. As shown in the table below her HbA1c was increasing prior to treatments. Her HbA1c dropped with treatments until March then started to rise again once she stopped her treatment regime.

| Date | HbA1c | Currents used and Frequency of treatment |
|---|---|---|
| July 2005 | 6.9 | No Treatment. |
| March 2006 | 7.0 | No Treatment. |
| November | 7.2 | Started treatments in October. |
| 2006 | | Currents received; 1 milliampere for 23 mins, |
| | | 60 microamperes 23-69 minutes, and |
| | | 400 nanoamperes 69-138 minutes. |
| | | She was having daily treatments from |
| | | October-December. |
| March 2007 | 6.3 | January-March. |
| | | Currents received; 1 milliampere for 23 |
| | | minutes, 60 microamperes for 23 minutes, |
| | | 200 nanoamperes 138 minutes. |
| | | She was having treatments approximately 3 |
| | | times per month. |
| June 2007 | 6.4 | No Treatments since March. |

| Time Period | average BSL | n |
|---|---|---|
| Oct-Dec 2006 | 10.3 | 3 |
| Jun-Oct 2007 | 6.6 | 4 |

### EXAMPLE 5

Initial Patient Status—A 77 year old woman with a history of type 2 diabetes, hypertension, hypercholesterolemia, hypothyroid (2̂ to Hashimoto's), and breast cancer.

She started her treatments in October 2006 and continued through until December 2006, on the BodiHealth TENS System. During treatments she was able to maintain her HbA1c, but as soon as she stopped her HbA1 started to rise again. Her BSL stabilized the longer she was on treatment and she noted that her early morning BSL were dropping below 6. She also noted that symptoms of peripheral neuropathy (burning feet), that had developed as a result of her diabetes, had resolved.

| Date | HbA1c | Currents used and Frequency of treatment |
|---|---|---|
| December 2005 | 7.8 | No Treatment. |
| April 2006 | 7.2 | No Treatment. |
| July 2006 | 7.3 | No Treatment. |
| November 2007 | 7.3 | Started treatments in October. |
| | | Currents received; 1 milliampere for 23 |
| | | mins, and 400 nanoamperes 138 minutes. |
| | | She was having treatments 3 times per week |
| | | most weeks from mid October-early |
| | | December. |
| January | 7.7 | No Treatments since December. |

| Time Period | average BSL | n |
|---|---|---|
| Oct-Nov 2006 | 7.8 | 12 |
| December 2007 | 6.9 | 4 |

### EXAMPLE 6

Initial Patient Status—A 70 year old woman with a history of type 2 diabetes (NIDDM), osteoarthritis, chronic pain and multiple operations.

She started her treatments in May 2006 and continued through until early October 2006, on the BodiHealth TENS System. She did not have any HbA1c tests taken before during or after this time. However she did have her BSL closely monitored and these steadily improved over time

| | average | |
|---|---|---|
| Time Period | BSL | n |
| May-Jul 2006 | 9.8 | 18 |
| Jul-Aug 2006 | 7.4 | 14 |
| Aug-Oct 2006 | 7.1 | 14 |

Currents used: 1 milliampere 23 minutes, 400 nanoamperes 138 minutes, very occasionally a 60 microampere current was added for 23-69 minutes.

She has treatments approximately twice a week throughout this time.

### EXAMPLE 7

Initial Patient Status—A 68 year old gentleman with had a history of type 2 diabetes (NIDDM), hypertension, stroke (CVA), chronic pain and polio.

He started his treatments at the end of August 2006 and continued through until the start of February 2007, on the BodiHealth TENS System. While receiving treatment his HbA1c level fell to 6.6. Of note after stopping treatments his HbA1c increased back up again to 7.8 in June 2007.

| Date | HbA1c | Currents used and Frequency of treatment |
|---|---|---|
| December 2005 | 7.8 | No Treatment. |
| April 2006 | 7.2 | No Treatment. |
| July 2006 | 7.3 | No Treatment. |
| October 2006 | 6.6 | Started treatments in August. |
| | | Currents received; 1 milliampere for 23 mins, 2 |
| | | milliampere for 23 mins, 60 microamperes for |
| | | 23-46 minutes, and 400 nanoamperes |
| | | 138 minutes. |
| | | He was having treatments 3 times per week |
| | | most weeks from August through to February. |
| June 2007 | 7.8 | No Treatments since February 2007. |

The foregoing Examples have been provided to further define, describe and illustrate some of the preferred embodiments of this invention and to facilitate the practice thereof. Notwithstanding, it is not the purpose of such Examples to define the scope of this invention, which has been reserved for the following claims.

What is claimed is:

1. A method for treating a patient suffering diabetes (Type I-Diabetes), or pre-diabetes (Type II-Diabetes), comprising delivering a therapeutic effective amount of an alternative direct micro-current to the patient, so as to abate one or more biological processes responsible for contributing to an insulin deficiency in said patient.

2. The method as claimed in claim 1 wherein the micro current is within the range of from 20 milli-amperes to 1 atto-ampere (1×10⁻¹⁸ amps).

3. The method as claimed in claim 1 wherein the micro current is within the range from 3 milli-amperes to 100 atto-amperes.

4. The method as claimed in claim 1 wherein the micro current is an alternating current having a frequency in the range of from 0.00065 Hz to 0.00085 Hz.

5. The method as claimed in claim 1 wherein the micro current is delivered to the patient by using an electrode wrap.

6. The method as claimed in claim 1 wherein the micro current has a duty cycle of 0.000732 Hz or one cycle every 22.77 minutes.

7. The method as claimed in claim 1 wherein treatment of a patient comprises applying one or more cycles of a current of 2 milliamps, with each cycle lasting for approximately 23 minutes, and further cycles being conducted at 300 nanoamps.

8. A method for preventing mitochondrial oxidative damage in a person comprising delivering a therapeutic effective amount of an alternative direct micro-current to the patient so as to abate one or more biological processes responsible for contributing to mitochondrial oxidative damage in said patient.

9. The method as claimed in claim 8, wherein the micro current comprises a current falling within the range of from 20 milli-amperes to 1 atto-ampere (1×10⁻¹⁸ amps).

10. The method as claimed in claim 8, wherein the micro current falls within the range from 3 milli-amperes to 100 atto-amperes.

11. The method as claimed in claim 8, wherein the micro current is an alternating current having a frequency in the range of from 0.00065 Hz to 0.00085 Hz.

12. The method as claimed in claim 8, wherein the micro current is delivered to the patient by using an electrode wrap.

13. The method as claimed in claim 8, wherein the micro current has a duty cycle of 0.000732 Hz or one cycle every 22.77 minutes.

14. The method as claimed in claim 8, wherein treatment of a patient comprises applying one or more cycles of a current of 2 milliamps, with each cycle lasting for approximately 23 minutes, and further cycles being conducted at 300 nanoamps.
